(19) United States
(12) Patent Application Publication
(10) Pub. No.: US 2026/0266785 A1
(43) Pub. Date: Sep. 10, 2026

(54) METHOD FOR QUANTIFYING THE PYROGENIC CARBON PRESENT IN A SAMPLE OF AN ORGANO-MINERAL OR MINERAL MATRIX COMPRISING CHARCOAL AND/OR BIOCHAR

(71) Applicant: IFP Energies nouvelles, RUEIL-MALMAISON (FR)

(72) Inventors: David SEBAG, RUEIL-MALMAISON CEDEX (FR); Marie-Liesse AUBERTIN, RUEIL-MALMAISON CEDEX (FR)

(21) Appl. No.: 19/167,884

(22) PCT Filed: Mar. 15, 2024
(86) PCT No.: PCT/EP2024/057079

§ 371 Date: Sep. 23, 2025

(30) Foreign Application Priority Data
Apr. 6, 2023 (FR) FR2303435

(51) Int. Cl. G01N 31/12; G01N 33/24

(52) U.S. Cl. CPC G01N 31/12; G01N 33/24

(57) ABSTRACT

The present invention relates to a process for quantifying the pyrogenic carbon content of a sample comprising an organo-mineral or mineral matrix and also charcoal and biochar. The process involves heating the sample under an inert atmosphere, followed by heating under an oxidizing atmosphere of the residue from the heating under an inert atmosphere. A Gaussian deconvolution is then applied to the curve for measuring the amount of CO₂ released as a function of temperature during heating under an oxidizing atmosphere, and determination is made of a first and a second Gaussian centered, respectively, on a first temperature between 380° C. and 540° C., which is preferably between 415° C. and 425° C., and very preferentially equal to 420° C., and a second temperature between 500° C. and 600° C., preferably between 570° C. and 580° C., and preferentially equal to 576° C. The pyrogenic carbon content is determined from the surface areas of the first and second Gaussians.

### CROSS-REFERENCE TO RELATED APPLICATIONS

This application is a 371 National Phase application under 35 U.S.C. § 371 of PCT/EP2024/057079 filed Mar. 15, 2024, and French Patent Application 2303435, filed Apr. 6, 2023, which are incorporated herein by reference in their entirety.

### BACKGROUND OF THE INVENTION

### Field of the Invention

The present invention may relate in particular to the field of soil science, agronomy and the environment, and more generally to any field concerned with the quantification of at least one of biochar and/or charcoal present in a mineral or an organo-mineral matrix.

The present invention may, for example, relate to the field of biochar production and marketing, in particular when sold in the form of a mixture with an organo-mineral or mineral matrix, for use, for example, as an organic soil improver (biochar-sediment) or as a building material, like hybrid green concrete (biochar-calcium carbonate) or simply by mixing biochar with concrete (mineral matrix).

### Description of the Prior Art

In the environmental field, in order to meet environmental challenges and notably to limit global warming to 1.5° C. compared to pre-industrial times, it is necessary to achieve the “Net Zero Emissions” objective by 2050. To achieve this, it is necessary not only to drastically reduce CO₂ emissions, but also to develop negative emission technologies. Biochar, a product of biomass pyrolysis, constitutes a negative emission technology, as it is a carbon-rich, biologically stable material. Moreover, in recent decades, pyrogenic organic matter has been recognized as an important component of very fertile Amazonian soil. Specifically, this material may have beneficial effects on soil fertility, in particular for water and nutrient retention and a liming effect. The name given to pyrolyzed organic matter differs as a function of its end use. The term “biochar” is used when the organic matter is pyrolyzed with the object of amending the soil to improve its properties. In the literature, “black carbon” describes the volatile components resulting from the incomplete combustion of biomass. In certain studies, the term “black carbon” is also used to describe a wider range of materials, forming a continuum ranging from charcoal to soot. The term “char” or “charcoal” or sometimes “wood charcoal” is used when organic matter is deliberately pyrolyzed, for example for use as a fuel or filter. It should be noted that charcoal differs from geological coal or coal of geological origin in the way it is produced, and also in its very different chemical and physical properties. Unlike charcoal derived from the pyrolysis of biomass, geological coal (for example hard coal) is produced by geological processes under high pressure and high temperature, over a longer timescale (several thousand years). Geological coal notably does not contain any pyrogenic carbon.

Thus, forms of charcoal other than geological coal (i.e. charcoal, char or biochar) may be present in a soil due to several contexts, such as vegetation fires and intentional amendment to improve soil properties. As charcoal is rich in biologically stable carbon (i.e. aromatic compounds), its quantification can be carried out through the pyrogenic carbon of which it is predominantly formed. There are many advantages to being able to quantify pyrogenic carbon in soil, depending on the context in which it has been introduced. In the case of vegetation fires, the pyrogenic carbon content constitutes a marker of fire frequency. This marker is particularly useful for archaeological studies, since high fire frequencies on the same site may attest to previous human occupation, while lower intensities are more likely to be the result of fires of natural origin. In the case of biochar amendments, quantifying the pyrogenic carbon in an amended plot enables monitoring of the remaining biochar content in the soil. Specifically, biochar may be readily subject to physical degradation. Moreover, quantifying the pyrogenic carbon outside the amended plot allows monitoring of the biochar's fate in the environment, notably its deposition zones.

The following documents will be cited during the description:
Arroyo-Kalin, M. A. (2008). Steps Towards an Ecology of Landscape: A Geoarchaeological Approach to the Study of Anthropogenic Dark Earths in the Central Amazon Region, Brazil (Doctoral dissertation, University of Cambridge).
Aubertin, M. L. (2022). Biochar-Compost Mixtures: Interactions and Impact on Carbon Sequestration and Soil Fertility (Doctoral dissertation, Sorbonne University).
Behar, F., Beaumont, V., & Penteado, H. D. B. (2001). Rock-Eval 6 Technology: Performances and Developments. Oil & Gas Science and Technology, 56(2), 111-134.
Chalk, P., & Smith, C. J. (2022). 13C Methodologies for Quantifying Biochar Stability in Soil: A Critique. European Journal of Soil Science, 73(3), e13245.
Cuypers, C., Grotenhuis, T., Nierop, K. G., Franco, E. M., de Jager, A., & Rulkens, W. (2002). Amorphous and Condensed Organic Matter Domains: The Effect of Persulfate Oxidation on the Composition of Soil/SedimentOorganic Matter. Chemosphere, 48(9), 919-931.
Glaser, B., Haumaier, L., Guggenberger, G., & Zech, W. (1998). Black Carbon in Soils: The Use of Benzenecarboxylic Acids as Specific Markers. Organic Geochemistry, 29(4), 811-819.
Llorente, M., Turrión, M. B., & Glaser, B. (2018). Rapid and Economical Quantification of Black Carbon in Soils Using a Modified Benzene Polycarboxylic Acids (BPCA) Method. Organic Geochemistry, 115, 197-204.
Paterson, G. A., & Heslop, D. (2015). New Methods for Unmixing Sediment Grain Size Data. Geochemistry, Geophysics, Geosystems, 16(12), 4494-4506.
Poot, A., Quik, J. T., Veld, H., & Koelmans, A. A. (2009). Quantification Methods of Black Carbon: Comparison of Rock-Eval Analysis with Traditional Methods. Journal of Chromatography A, 1216(3), 613-622.
Sebag, D., Disnar, J. R., Guillet, B., Di Giovanni, C., Verrecchia, E. P., & Durand, A. (2006). Monitoring Organic Matter Dynamics in Soil Profiles by “Rock-Eval Pyrolysis”: Bulk Characterization and Quantification of Degradation. European Journal of Soil Science, 57(3), 344-355.
Sebag, D., Garcin, Y., Adatte, T., Deschamps, P., Menot, G., & Verrecchia, E. P. (2018). Correction for the SideriteEeffect on Rock-Eval Parameters: Application to the Sediments of Lake Barombi (southwest Cameroon). Organic Geochemistry, 123, 126-135.
Simpson, M. J., & Hatcher, P. G. (2004). Overestimates of Black Carbon in Soils and Sediments. Naturwissenschaften, 91(9), 436-440.

Various techniques are known for quantifying pyrogenic carbon in soil or sedimentary material, based on chemical, magnetic, optical or thermal differences between soil and charcoal, or on the presence of molecular markers.

A commonly used technique is quantification by extraction of benzene polycarboxylic acids (BPCA) following chemical oxidation of aromatic structures, by gas chromatographic analysis, as described for example in (Glaser et al., 1998), or else by elemental analysis, as described for example in (Llorente et al. 2018). However, extraction is time-consuming and may add bias to BPCA quantification.

The use of natural carbon isotopy is a direct, accurate and reproducible quantification method, allowing the source of a carbon from a mixture of two carbon components with significantly different isotopic (δ13C) signatures to be distinguished, as described for example in (Aubertin et al., 2022). However, isotopic analysis may only be applied in the case of a charcoal-soil mixture, in which the two components have significantly different isotopic signatures. Isotopic enrichment methods can also be used to quantify pyrogenic carbon, but this involves (time-consuming) incubation and the results may be biased by the nonuniform distribution of labeled carbon added to the plant, as described for example in (Chalk and Smith, 2022).

Other methods for quantifying charcoal are based on the observation of differences in color or density of charcoal particles from a microscope photograph, as described for example in (Arroyo-Kalin, 2008). However, these methods are time-consuming (preparation time for thin slides and sample handling), only take into account particles above a certain diameter, are not very reproducible as they are manipulator-dependent, and only approximate the mass of the charcoal, based on a surface count.

Among thermal methods, the thermochemical oxidation method involves a chemical oxidation pretreatment with an acid to remove inorganic carbon, then separation of pyrogenic and non-pyrogenic carbon with combustion at temperatures of about 350° C. for at least 2 hours, followed by residual carbon analysis by 13C NMR or elemental analysis, as described for example in (Poot et al. 2009). In addition to the time-consuming aspect of this method, it may also lead to overestimates of pyrogenic carbon due to the formation of pyrogenic carbon during the combustion phase, as described for example in (Simpson and Hatcher, 2004). Thermogravimetric analysis measures several emission peaks during heating, but it is nevertheless difficult to differentiate emission peaks related to pyrogenic carbon and soil components with this method, as described for example in (Cuypers et al., 2002). One method for quantifying pyrogenic carbon in soil is the differential scanning calorimetry (DSC) technique, in which the sample causes changes in the flow of heat as a function of the temperature rise gradient. A close correlation may be made between variations in the flow of heat and the amount of carbon. To quantify the pyrogenic carbon, it suffices to differentiate between the amount of carbon above a threshold temperature, around 400° C., in a sample of the same soil/sediment with and without carbon. However, DSC is an indirect measurement of stable carbon in a sample, which may introduce inaccuracy in carbon quantification.

Thus, despite its obvious interest, the quantification of pyrogenic carbon in soil often remains difficult to perform, and the various existing techniques have drawbacks due to, for example, their price, analysis time, accuracy, or the fact that they are not always reproducible.

Methods for the thermal analysis of the organic matter of soils which rely on measurements of amounts of hydrocarbon (HC) compounds, of carbon monoxide (CO) and/or of carbon dioxide (CO₂) released over time by a sample subjected to a temperature sequence under an inert atmosphere (pyrolysis phase) and then/or to a temperature sequence under an oxidizing atmosphere (oxidation phase) are also known. These methods were initially developed in the field of the petroleum industry, for the purposes of characterizing the organic fraction of sedimentary rocks. For example, the “ROCK-EVAL® BULK ROCK” method, initially developed in the context of conventional source rock samples, is known to distinguish pyrolyzed organic carbon from refractory organic carbon (Behar et al., 2001). The document (Poot et al., 2009) describes that the amount of refractory carbon measured in such a thermal analysis can be used to approximate the quantification of pyrogenic carbon in a soil or sediment sample. More specifically, this document describes that the “ROCK-EVAL® BULK ROCK” method allows pyrolyzable carbon (PC) to be readily and quickly separated from residual carbon (RC). RC corresponds to refractory organic carbon, derived from organic matter that is thermally resistant to the pyrolysis phase and is oxidized during the oxidation phase. The document thus proposes to approximate RC as a measure of pyrogenic carbon, which it calls “black carbon” and defines as a continuum ranging from pyrolyzed biomass charcoal to soot. However, this latter variant remains imprecise, due to the fact that RC may also be partly produced during the pyrolysis phase. Thus, the pyrogenic carbon analyzed with this method may be slightly overestimated.

### SUMMARY OF THE INVENTION

The present invention allows these drawbacks to be overcome. In particular, the present invention allows rapid and accurate quantification of pyrogenic carbon in a sample of an organo-mineral or mineral matrix such as soil, by means of thermal analysis, notably comprising analysis of carbon emissions during an oxidation phase of the sample.

The present invention relates to a process for quantifying the pyrogenic carbon content present in a sample comprising an organo-mineral or mineral matrix and also at least one of charcoal and biochar. The process according to the invention comprises at least the following steps:
A) the sample is heated under an inert atmosphere according to a first temperature sequence, with an initial temperature (T0) of between 10° and 300° C., preferentially equal to 200° C., and a final temperature (TF) of between 50° and 800° C., preferentially equal to 650° C.;
B) a residue of the sample resulting from the heating under an inert atmosphere is heated under an oxidizing atmosphere according to a second temperature sequence with an initial temperature (T0′) of between 10° and 300° C., preferentially equal to 200° C., and a final temperature (TF′) of between 70° and 1000° C., preferentially equal to 850° C., the second temperature sequence comprising at least one thermal gradient between 1° C. and 50° C./min, preferentially between 15° C./min and 35° C./min, very preferentially equal to 25° C./min, and measuring at least one amount of CO₂ (QCO2) released during the second temperature sequence;
C) starting from a curve representing the evolution, as a function of temperature, of the amount of CO₂ released during the heating under an oxidizing atmosphere, a Gaussian deconvolution is applied to the curve, so as to determine a first and a second Gaussian centered, respectively, on a first and a second temperature, the first temperature being between 380° C. and 540° C., preferably between 415° C. and 425° C., very preferentially equal to 420° C., and the second temperature being between 500° C. and 600° C., preferably between 570° C. and 580° C., preferentially equal to 576° C.;
D) from the surface areas of the first and second Gaussians, the pyrogenic carbon content X₃ present in the sample is determined according to a formula of the type:

$${X}_{3} = \frac{{X}_{2} - A \cdot {X}_{1}}{1 + A \cdot B}$$

in which X₁ and X₂ are carbon contents determined respectively from the surface areas of the first and second Gaussians, A is a coefficient representing the proportion of the matrix in the second Gaussian relative to the first Gaussian, and B is a coefficient representing the proportion of at least one of the charcoal and the biochar in the first Gaussian relative to the second Gaussian.

According to an implementation of the invention, the first temperature sequence may comprise an isothermal steady stage of predetermined duration at the initial temperature (T0) of the first temperature sequence, followed by a thermal gradient to reach the final temperature (TF) of the first temperature sequence, the predetermined duration of the isothermal steady stage of the first temperature sequence possibly being between 1 and 5 minutes, and possibly preferentially equal to 3 minutes, and the thermal gradient of the first temperature sequence possibly being between 1° C./min and 50° C./min, preferentially between 15° C./min and 35° C./min, possibly being very preferentially equal to 25° C./min.

According to an implementation of the invention, the second temperature sequence may also comprise an isothermal steady stage of a predetermined duration at a temperature of between 50° and 600° C., preferably between 57° and 580° C., preferentially equal to 576° C., the predetermined duration of the isothermal steady stage of the second temperature sequence possibly being between 1 and 5 minutes, possibly being preferentially equal to 3 minutes.

According to an implementation of the invention, a sample of the pure organo-mineral or mineral matrix may also be available, and the coefficient A of step D) may be determined beforehand in the following manner: steps A) to C) are applied to the sample of the pure organo-mineral or mineral matrix, and the coefficient A is determined by calculating the ratio between the surface area of a second Gaussian and the surface area of a first Gaussian determined from the sample of the pure organo-mineral or mineral matrix.

According to an implementation of the invention, a sample of at least one of the pure biochar and the pure charcoal may also be available, and the coefficient B of step D) may be determined beforehand in the following manner: steps A) to C) are applied to the sample of at least one of the biochar and the charcoal, and coefficient B is determined by calculating the ratio between the surface area of the first Gaussian and the surface area of the second Gaussian determined from the sample of pure biochar and charcoal.

According to an implementation of the invention, step D) may be applied by use of a coefficient A ranging from a value of 0.17 to a value of 0.73, preferentially equal to at least one of 0.19, and by use of a coefficient B ranging from a value of 0.10 to a value of 4.98, preferentially equal to 0.32.

According to an implementation of the invention, a total mass of pyrogenic carbon Qc,bc_mix present in the sample may be determined according to a formula of the type: Qc,bc_mix=X3/C*K, where C is a ratio between a carbon content determined from a surface area of a second Gaussian determined for at least one of a sample of pure biochar and charcoal, and a total mass of carbon in at least one of the sample of pure biochar and charcoal, and where K is a multiplicative coefficient between 12.0 and 12.5, and preferentially equal to 12.2.

Alternatively, a total mass of pyrogenic carbon Qc,bc_mix present in the sample may be determined according to a formula of the type: Qc,bc_mix=X3/C*K, in which C is a ratio between a value of 0.51 and a value of 1.76, preferentially equal to 1.06, and in which K is a multiplication coefficient between 12.0 and 12.5, preferentially equal to 12.2.

Other characteristics and advantages of the process according to the invention will become apparent on reading the description below of nonlimiting implementation examples, with reference to the appended figures described below.

### BRIEF DESCRIPTION OF THE DRAWINGS

FIG. 1A schematically illustrates the evolution of temperature as a function of time in the temperature sequence of the first step of the process according to the invention.

FIG. 1B schematically illustrates the evolution of temperature as a function of time of a variant of the temperature sequence of the first step of the process according to the invention.

FIG. 2 shows, as an application example, the CO₂ curve measured during step 2) of the process according to the invention, and also the first and second Gaussians resulting from the Gaussian deconvolution according to step 3) of the process according to the invention.

FIG. 3 schematically shows the proportion of the organo-mineral or mineral matrix and at least one of the proportion of biochar and charcoal in the first Gaussian resulting from the Gaussian deconvolution according to step 3) of the process according to the invention, and also at least one of the proportion of biochar and charcoal and the proportion of the organo-mineral or mineral matrix in the second Gaussian resulting from the Gaussian deconvolution according to step 3) of the process according to the invention.

### DETAILED DESCRIPTION OF THE INVENTION

The invention relates to a process for quantifying the pyrogenic carbon present in a sample comprising at least one of an organo-mineral and a mineral matrix and also charcoal and biochar.

The term “pyrogenic carbon” means the organic fraction that has undergone pyrolysis, i.e. the effect of at least one of fire and heat (temperature >200° C.) in the absence of or in a low concentration of oxygen.

The term “organo-mineral or mineral matrix” means an unconsolidated, porous material formed of a mixture of at least one of organic and mineral particles of varying size and at least one of chemical and mineralogical composition.

The term “charcoal” means the solid residues of a chemical transformation under the effect of a rise in temperature, resulting from pyrolysis or incomplete combustion of plant or animal biomass.

The term “biochar” means charcoal produced with the intention of using it as an organic amendment, notably to improve the physicochemical properties of at least one of a soil and its carbon storage.

The process of the invention requires at least one sample comprising an organo-mineral and a mineral matrix and also charcoal and biochar.

According to an implementation of the invention, the sample comprising an organo-mineral or mineral matrix and also at least one of charcoal and biochar may be a soil sample comprising at least one of charcoal and biochar. The term “soil” means all the external layers of the Earth's surface formations. A soil sample may be collected manually in a pit or by coring using an auger.

According to an implementation of the invention, the sample comprising an organo-mineral or mineral matrix and also at least one of charcoal and biochar may be a sample of an amendment comprising an organo-mineral or mineral matrix and also at least one of charcoal and biochar.

According to an implementation of the invention, the sample comprising at least one of an organo-mineral and mineral matrix and also at least one of charcoal and biochar may be a soil sample, a sample of natural sediment or sediment polluted with fire residues, or else a sample of mineral materials (concrete, excavated earth, sediments) mixed with charcoal/biochar. The soil may be agricultural soil or anthrosol deliberately enriched with biochar.

Advantageously, the sample may be sieved using a sieve with orifices having a diameter of 2 mm, dried at a temperature below 40° C., and then ground until fragments having dimensions of less than 200 μm are obtained.

Preferably, a sample of the pure organo-mineral or mineral matrix, i.e. comprising neither biochar nor charcoal, is also available.

Advantageously, a sample of at least one of the biochar and charcoal present in the sample under consideration may also be available.

The process according to the invention may be advantageously but non-limitingly performed using the Rock-Eval® device (IFP Energies Nouvelles, France), as described in patents FR 2 227 797 (U.S. Pat. No. 3,953,171) and FR 2 472 754 (U.S. Pat. No. 4,352,673). Specifically, the Rock-Eval® device comprises at least:
a furnace for pyrolysis in a non-oxidizing atmosphere;
means for transferring the pyrolysis residues into an oxidation furnace;
a furnace for oxidation in an oxidizing atmosphere;
means for measuring the amount of hydrocarbon (HC) compounds released during the pyrolysis; and
means for measuring the carbon monoxide (CO) and the carbon dioxide (CO₂).

The process may also be performed using a single pyrolysis furnace, which can operate both in a non-oxidizing atmosphere and in an oxidizing atmosphere, interacting with a device for measuring the amount of hydrocarbon compounds released during the pyrolysis and a device for measuring the carbon monoxide and the carbon dioxide.

The process according to the invention comprises at least the following steps:
1) Heating sequence under an inert atmosphere (pyrolysis)
2) Heating sequence under an oxidizing atmosphere (oxidation)
3) Gaussian deconvolution
4) Determination of the pyrogenic carbon content

The steps of the process according to the invention are detailed below.

### 1. Heating Sequence Under an Inert Atmosphere (Pyrolysis)

During this step, the sample comprising an organo-mineral or mineral matrix and also at least one of biochar and charcoal is heated under an inert atmosphere (for instance a nitrogen or helium stream) according to a temperature sequence with an initial temperature (noted T0 hereinbelow) of between 10° and 300° C., preferentially equal to 200° C., and a final temperature (noted TF hereinbelow) of between 50° and 800° C., preferentially equal to 650° C.

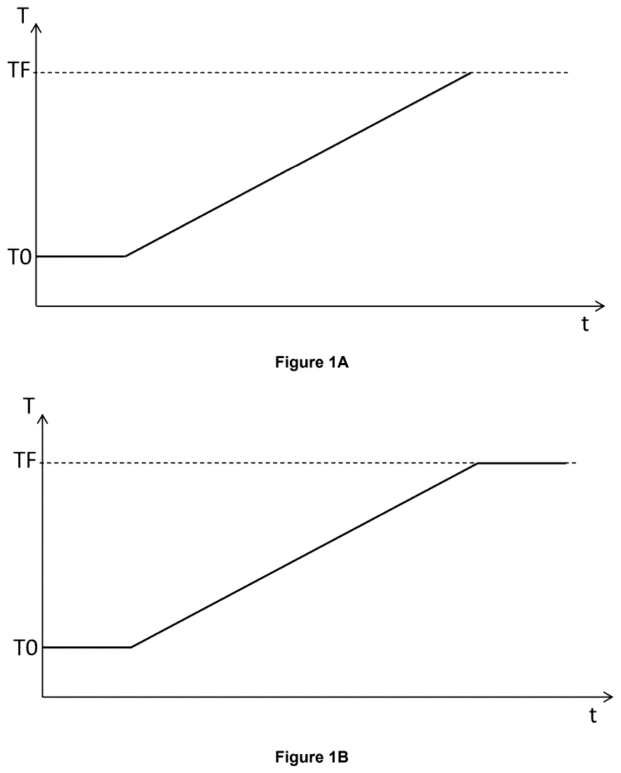

In a preferred manner, the temperature sequence under an inert atmosphere may comprise at least one isothermal steady stage at the initial temperature T0, followed by a predetermined thermal gradient so as to raise the sample temperature to the final temperature TF. FIG. 1A diagrammatically illustrates the change in the temperature T as a function of the time t of such a sequence of temperatures, having an isothermal steady stage at the temperature T0, followed by a thermal gradient up to the temperature TF.

Advantageously, the temperature sequence under an inert atmosphere of this embodiment may also comprise a second isothermal steady stage, at the final temperature TF. In other words, a second isothermal steady stage at the final temperature TF follows the phase of the temperature sequence appearing in the form of a thermal gradient. This makes it possible to continue, if necessary, the cracking of the compounds having a cracking temperature close to the final temperature TF of the temperature sequence under an inert atmosphere according to the invention. FIG. 1B schematically illustrates the change in the temperature T as a function of the time t of such a temperature sequence, having two isothermal steady stages, at the temperatures T0 and TF as defined above, connected together by a thermal gradient.

According to an implementation of the invention, the initial temperature T0 is preferably equal to 200° C. This temperature is indeed sufficient to release the most labile organic compounds present in most soil, organic amendment or sediment samples.

According to an implementation of the invention, the final temperature TF is preferably equal to 650° C., to avoid obtaining CO and CO₂ curves with incomplete peaks at the end of pyrolysis, measured notably on natural samples (fresh and dried plant tissues, litter, peat and plant composts, organo-mineral and mineral soils, surface formations).

According to an implementation of the invention, the isothermal steady stage(s) of the temperature sequence under an inert atmosphere may have a nonzero predetermined duration (for example, greater than half a minute), preferentially between 1 and 5 minutes, and very preferentially equal to 3 minutes. Such durations make it possible to regard the cracking of the compounds having a cracking temperature close to the temperature of the isothermal steady stage as being complete. According to the implementation of the invention according to which the temperature sequence under an inert atmosphere according to the invention comprises several isothermal steady stages and in particular two isothermal steady stages at the temperatures T0 and TF, the duration of one isothermal steady stage can be different from the duration of the other isothermal steady stage(s).

According to an implementation of the invention, the thermal gradient(s) of the temperature sequence under an inert atmosphere may be between 1 and 50° C./min, preferably between 15° and 35° C./min, and are preferably equal to 25° C./min. Such values constitute compromises allowing thermal cracking of the compounds, while at the same time limiting the duration of implementation of the process.

According to an implementation of the invention, it is possible to measure, continuously (i.e. continuously over time), an amount of hydrocarbon compounds released during heating under an at least one of inert atmosphere, and an amount of CO₂ and an amount of CO contained in an effluent resulting from the heating. In other words, during this sequence, the amount of HC, of CO and of CO₂ which are released by the sample by thermal cracking of the organic matter and by the thermal decomposition of the carbonate-based minerals can be continuously measured. The amount of hydrocarbon compounds can be measured by use of a detector of the flame ionization (FID) type. The amount of CO and of CO₂ which are released can be measured by means of a detector of the infrared (IR) type. As a variant, other means for measuring the amount of HC, of at least one of CO and of CO₂ can be used. According to this implementation, on conclusion of this step applied to a given sample, a first curve representative of the amount of hydrocarbon compounds which are released over time during the pyrolysis phase is obtained, along with two other curves representative of the amount of CO and CO₂ which are released over time during the pyrolysis phase. Such measurements can help to determine standard parameters for such a thermal analysis, in particular the parameter denoted TOC (Total Organic Carbon), which corresponds to the carbon content of the sample, determined from the amount of hydrocarbons released by the sample and from the amounts of CO and of CO₂ released below threshold temperatures during the pyrolysis phase and the oxidation phase, and the parameter denoted MinC (Mineral Carbon), which corresponds to the mineral carbon content of the sample, determined from the amounts of CO and of CO₂ released by the sample above threshold temperatures during the pyrolysis phase and the oxidation phase. A description of these general parameters can be found in the document (Behar et al., 2001).

Generally, this particular heating sequence under an inert atmosphere is sufficient to allow the thermal cracking of classes of compounds comprising mineral carbon and organic carbon, notably:
thermally very labile compounds, which are particularly abundant in fresh biological tissues and which are generally released at temperatures of between approximately 80° C. and 360° C.;
thermally labile compounds, which are predominant in organic samples, such as litter or peat, and which are generally released at temperatures of between approximately 360° C. and 420° C.;
thermally resistant compounds, which are predominant in organo-mineral samples (soils) or mineral samples (alluvial deposits, colluvial deposits) and which are generally released at temperatures of between approximately 420° C. and 470° C.;
thermally refractory compounds, which are generally released at temperatures of between approximately 470° C. and 520° C.;
and thermally very refractory compounds, which are present in greater proportions in decomposition residues or exogenous fractions, such as pyrogenic or petrogenic organic matter, and which are generally released at temperatures of between approximately 520° C. and 650° C.

According to an implementation of the invention, the temperature sequence under an inert atmosphere according to the invention can be preceded by a phase of rise in temperature of the pyrolysis furnace, which can be in the form of a thermal gradient, for example between 1 and 50° C./min, preferably between 2° and 25° C./min, or of any other form of curve of rise in temperature of the pyrolysis furnace. This preliminary phase of rise in temperature of the pyrolysis furnace makes it possible to bring the pyrolysis furnace to the initial temperature of the temperature sequence under an inert atmosphere according to the invention. This preliminary phase can contribute toward starting the thermal cracking of the compounds, the cracking temperature of which is less than the initial temperature of the temperature sequence under an inert atmosphere according to the invention, notably in the case of fresh biological tissues.

According to an implementation of the invention, the temperature sequence under an inert atmosphere according to the invention can be followed by a phase of lowering in temperature of the pyrolysis furnace, which can be in the form of a thermal gradient, for example between −1 and −50° C./min, preferably between −20 and −25° C./min, or of any other form of curve of lowering in temperature of the pyrolysis furnace. This terminal phase of lowering in temperature of the pyrolysis furnace makes it possible, if necessary, to complete the thermal cracking of the compounds which are associated with the final temperature of the temperature sequence under an inert atmosphere according to the invention.

### 2) Heating Sequence Under an Oxidizing Atmosphere (Oxidation)

In this second step, the solid residue of the sample obtained on conclusion of the sequence of heating under an inert atmosphere as described in step 1 above is subjected to oxidation according to a predefined temperature sequence, with an initial temperature (referred to hereinbelow as T0′) of between 10° and 300° C., preferentially equal to 200° C., and a final temperature (referred to hereinbelow as TF′) of between 70° and 1000° C., preferably equal to 850° C. (so as to deplete the mineral carbon stock). Furthermore, according to the invention, the temperature sequence of this heating under an oxidizing atmosphere comprises at least one thermal gradient of between 1 and 50° C./min, preferably between 15° and 35° C./min, preferably equal to 25° C./min.

In general, the preferential temperature range for the initial temperature T0′ of the temperature sequence under an oxidizing atmosphere helps to avoid episodes of instantaneous combustion of the sample residue at the start of the oxidation cycle.

According to an implementation of the invention, the temperature sequence under an oxidizing atmosphere can also comprise an isothermal steady stage at the initial temperature T0′ of a predetermined nonzero duration (for example greater than half a minute), and may preferentially be between 1 and 5 minutes, very preferentially 3 minutes.

According to the invention, at least an amount of CO₂ (and optionally an amount of CO) released during this second temperature sequence is measured continuously. According to an implementation of the invention, this measurement may be performed using an infrared (IR) sensor. It should be noted that such a sensor provides values measured in millivolts (mV). In a conventional manner, an amount of CO₂ released during this second temperature sequence, noted as Xtot hereinbelow, is determined by determining an area under the curve measured (optionally between predefined temperatures) by this sensor, according to a formula of the type:

$$Xtot = \frac{12}{44.} \cdot \frac{S u r f c}{mass} \qquad ( 1 )$$

in which SurfC corresponds to the area under the curve (also known as the thermogram) representing the amount of CO₂ released during this second temperature sequence, mass corresponds to the mass of the sample, and in which Xtot is expressed as mg/g of sample. As a variant, other means of measuring the amount of CO₂ may be used.

Advantageously, the temperature sequence under an oxidizing atmosphere may also comprise an isothermal steady stage at a temperature of between 50° and 600° C., preferably between 57° and 580° C., preferentially equal to 576° C. This isothermal steady stage makes it possible to better separate, in a curve representing the temperature-dependent evolution of the amount of CO₂ released during heating under an oxidizing atmosphere, a component attributed to biochar or charcoal from a component attributed to the mineral or organo-mineral matrix present in the sample under consideration. This notably allows the result of step 3) of the process according to the invention described below to be improved. This isothermal steady stage may be of a predetermined nonzero duration (for example greater than half a minute), preferentially between 1 and 5 minutes, very preferentially 3 minutes. According to this embodiment, the temperature sequence under an oxidizing atmosphere can also comprise an additional thermal gradient (i.e. in addition to the at least one thermal gradient of the temperature sequence of step 2) of the process according to the invention), of between 1° C. and 50° C./min, preferably between 15 and 35° C./min, very preferentially equal to 25° C./min. Thus, according to this implementation, the isothermal steady stage at a temperature of between 50° and 600° C., preferably between 57° and 580° C., preferentially equal to 576° C., may be both preceded and followed by the at least two thermal gradients of this embodiment.

### 3) Gaussian Deconvolution

During this step, starting from a curve representing the evolution, as a function of temperature, of the amount of CO₂ released during the heating under an oxidizing atmosphere, a Gaussian deconvolution is applied to the curve, to determine (at least) a first and a second Gaussian centered, respectively, on a first and a second temperature, the first temperature being between 380° C. and 540° C., preferably between 415° C. and 425° C., very preferentially equal to 420° C., and the second temperature being between 500° C. and 600° C., preferably between 570° C. and 580° C., preferentially equal to 576° C.

The term “Gaussian deconvolution” means the breaking down of a curve (in this case the curve representing the evolution, as a function of temperature, of the amount of CO₂ released during the second temperature sequence) into elementary components, each corresponding to a Gaussian distribution.

Thus, this step aims to approximate the curve representing the evolution, as a function of temperature, of the amount of CO₂ released during the second temperature sequence by two Gaussians, more precisely, a first Gaussian centered on a temperature between 380° C. and 540° C., preferably between 415° C. and 425° C., very preferentially equal to 420° C., and a second Gaussian centered on a temperature between 500° C. and 600° C., preferably between 570° C. and 580° C., preferentially equal to 576° C. Specifically, the temperature range of the first Gaussian, between 380° C. and 540° C., is characteristic of the temperature range of the main peak of a curve representing the amount of CO₂ released by a sample of pure mineral or organo-mineral matrix, irrespective of the type of mineral or organo-mineral matrix, as described for example in the document (Sebag et al. 2018). Specifically, it has been shown that for this type of sample (mineral or organo-mineral matrix), the largest CO₂ peak is emitted at such temperatures during the oxidation phase. These temperatures are lower than those at which the maximum CO₂ is emitted for biochars or charcoals, due to the lower thermal stability of the mineral or organo-mineral matrix. Moreover, the temperature range of the second Gaussian, between 50° and 600° C., is characteristic of the temperature range of the peak of a curve representing the amount of CO₂ released by a sample of at least one of pure charcoal and biochar, irrespective of the type of at least one of charcoal and biochar, as described for example in the document (Aubertin et al., 2022). Specifically, it has been shown that biochar or charcoal samples emit a majority of CO₂ over this temperature range. In other words, the temperatures of the peaks of the two Gaussians according to the invention are, as it were, “signatures” of the mineral or organo-mineral matrices and at least one of the charcoal and biochar, irrespective of their origin and composition.

According to a nonlimiting implementation of the invention, the residual Gaussian deconvolution method described in the document (Sebag et al., 2006) may be used. More precisely, this method gradually subtracts the Gaussians centered on the signal's main peaks.

According to another implementation, the Gaussian deconvolution method can be used by an End-Member Mixing Analysis notably described in the document (Paterson and Heslop, 2015), and which determines, by use of an algorithm, the Gaussian components that best describe the signal, with a given number of Gaussians.

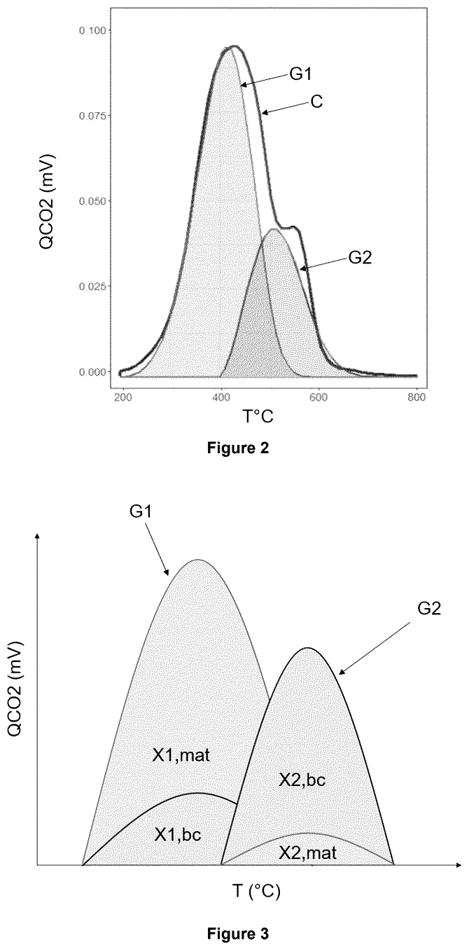

FIG. 2 illustrates the result of breaking down a curve C measuring the amount of CO₂ QCO2 released during step 2) of the process according to the invention into two Gaussians G1, G2.

### 4) Determination of the Pyrogenic Carbon Content

In this step, the pyrogenic carbon content in the sample under consideration is determined from the surface areas of the first and second Gaussians determined in step 3). More specifically, according to the invention, the pyrogenic carbon content present in the sample under consideration is determined, noted as X₃ hereinbelow, according to a formula of the type:

$${X}_{3} = \frac{{X}_{2} - A \cdot {X}_{1}}{1 + A \cdot B} \qquad ( 2 )$$

in which X₁ and X₂ are carbon contents determined respectively from the surface areas of the first and second Gaussians, A is a coefficient representing the proportion of the matrix in the second Gaussian relative to the first Gaussian, and B is a coefficient representing the proportion of at least one of charcoal and the biochar in the first Gaussian relative to the second Gaussian.

According to an implementation of the invention, X₁ can be determined according to the formula:

$${X}_{1} = SurfG 1 \qquad ( 3 )$$

and X₂ can be determined according to the formula:

$${X}_{2} = S u rfG 2 \qquad ( 4 )$$

in which SurfG1 and SurfG2 are, respectively, the surface areas of the first and second Gaussians determined on conclusion of step 3 of the process according to the invention.

Equation (2) arises from the fact that the deconvolution is imperfect for separating the contribution of the organo-mineral or mineral matrix from at least one of the contribution of biochar and charcoal in a curve measuring the amount of CO₂ released during an oxidation phase. In other words, the first Gaussian resulting from the deconvolution according to the invention does indeed comprise a predominant contribution linked to the organo-mineral or mineral matrix (noted as X_{1,mat} hereinbelow), but also includes a contribution from at least one of biochar and charcoal (noted as X_{1,bc} hereinbelow). Similarly, the second Gaussian resulting from the deconvolution according to the invention does indeed comprise a predominant contribution from at least one of biochar and charcoal (noted as X_{2,bc} hereinbelow), but also includes a contribution linked to the organo-mineral or mineral matrix (noted as X_{1,bc} hereinbelow). This is notably illustrated in FIG. 3, which schematically represents the proportion of organo-mineral or mineral matrix X1, mat and at least one of the proportion of biochar and charcoal X_{1,bc} in the first Gaussian G1, and also at least one of the proportion of biochar and charcoal X_{2,bc} and the proportion of organo-mineral or mineral matrix X2,mat in the second Gaussian G2. With these notations, coefficients A and B according to the invention may be described by the following formulae:

$$A = \frac{{X}_{2 , mat}}{{X}_{1 , mat}} \qquad ( 5 )$$

$$and$$

$$B = \frac{{X}_{1 , bc}}{{X}_{2 , bc}} \qquad ( 6 )$$

According to a first variant of the invention, at least one of coefficient A and coefficient B may be determined from a sample of at least one of the pure organo-mineral and mineral matrix and at least one of a sample of pure biochar and charcoal respectively, representative of at least one of the organo-mineral and mineral matrix and at least one of the biochar and charcoal present in the sample under consideration, to which steps 1), 2) and 3) described above are applied. Coefficient A may thus be determined by the ratio between at least one of the surface area of the second Gaussian and the surface area of the first Gaussian determined from at least one of the sample of pure organo-mineral and mineral matrix. Coefficient B may be determined by the ratio between the surface area of the first Gaussian and the surface area of the second Gaussian determined from at least one of the pure biochar and charcoal sample.

According to a second variant of the invention, and notably if samples of the pure organo-mineral and matrix and at least one of pure biochar and charcoal, representative of the organo-mineral or mineral matrix and at least one of the biochar and charcoal present in the sample under consideration, are not available, equation (2) above can be used by use of a coefficient A between a value of 0.17 and a value of 0.73, preferentially equal to 0.19, and at least one of a coefficient B between a value of 0.10 and a value of 4.98, preferentially equal to 0.32. These ranges and preferential values of coefficients A and B were determined from a plurality of samples of pure organo-mineral or mineral matrix and samples of at least one of pure biochar and charcoal, of different types, to which the method described above was applied. In particular, samples of at least one of pure organo-mineral and mineral matrix of soil and sediment type from various climatic conditions and with various total organic carbon contents, and samples of pure biochar and/or charcoal of various plant biomass and various pyrolysis temperatures, between 450° C. and 650° C., were used. The preferential value of coefficients A and B corresponds to the median of the values thus determined for the plurality of samples.

Thus, on conclusion of this step, the pyrogenic carbon content present in the sample under consideration, comprising both a mineral or organo-mineral matrix and at least one of charcoal and biochar, is obtained.

According to an implementation of the invention, the total mass of pyrogenic carbon present in the sample under consideration, noted as Qc,bc_mix hereinbelow, may be determined according to a formula of the type:

$$Q c , bc\_mix = X 3 / C * K \qquad ( 7 )$$

in which
   C is the ratio between the carbon content determined from the surface area of the second Gaussian determined in the case of a sample of at least one of pure biochar and charcoal, noted as X2, bc, and the total mass of carbon in the sample of at least one of pure biochar and charcoal; in other words, the ratio C can be written as follows

$$C = X 2 , bc / ( TOC , bc * Qty , bc ) \qquad ( 8 )$$

in which TOC,bc and Qty,bc respectively correspond to the total organic carbon and the total mass of a pure biochar/charcoal sample. According to an implementation of the invention, and notably if at least one of a pure biochar and charcoal sample, representative of the biochar and charcoal present in the sample under consideration, is not available, equation (2) above can be used by means of a ratio C between a value of 0.51 and a value of 1.76, preferentially equal to 1.06. This range and this preferential value were determined from a plurality of different types of at least one of pure biochar and charcoal samples. The preferential value corresponds to the median of the values thus determined for the plurality of samples.
   K is a multiplicative coefficient. According to an implementation of the invention, the coefficient K may be between 12.0 and 12.5, and is preferentially equal to 12.2. Such values were determined from a plurality of different types of at least one of pure biochar and charcoal samples.

### Examples

The characteristics and advantages of the process according to the invention will become more clearly apparent on reading the application example below.

The present invention is applied to determine the mass of pyrogenic carbon present in a sample corresponding to a soil-biochar mixture.

A plurality of samples are generated, for different mass ratios between biochar and soil, by homogeneous mixing between a soil of agricultural origin, and an industrial biochar of herbaceous plants. The total organic carbon (TOC) of the biochar and the soil is 82.60% and 5.55%, respectively. Each sample is dried at a temperature of less than or equal to 40° C. until its weight has stabilized, and is then ground to below 200 μm.

Each sample is heated under an inert atmosphere according to the invention, and its residue is then heated under an oxidizing atmosphere according to the invention. FIG. 2 shows curve C of the amount of CO₂ QCO2 measured during step 2) of the process according to the invention, and also the result of step 3) of the process according to the invention, in the form of two Gaussians G1, G2 in the case of a sample containing 1% by mass of biochar.

Table 1 shows the mass of pyrogenic carbon (given in mg of carbon, mgC) present in the samples under consideration, determined on conclusion of step 4 of the process according to the invention applied according to the first variant described above (determination of coefficients A and B of equation (2) from pure soil and biochar samples; column Qc,bc_mix_V1) and according to the second variant described above (determination of coefficients A and B of equation (2) from their preferred values defined above; column Qc,bc_mix_V2), and also the actual mass of pyrogenic carbon present in the samples under consideration (column Qc,bc_mix_REAL), as a function of their mass ratio between biochar and soil (Ratio column). It can be seen that the mass of pyrogenic carbon determined by the present invention, applied according to its first variant or its second variant, are very close to the actual values (average error of 2.31% and maximum of 76.45% (sample with a lower content of biochar/charcoal) for the first variant; average error of −3.54% and maximum of 29.63% for the second variant).

These results were obtained in less than 90 minutes for each sample, which essentially corresponds to the heating time under an inert atmosphere and the heating time under an oxidizing atmosphere for each sample.

Thus, the present invention makes it possible to quickly and accurately quantify the pyrogenic carbon in a sample of an organo-mineral or mineral matrix also comprising biochar or charcoal, by use of a thermal analysis that is simple to perform.

| | Qc, bc_mix_REAL | Qc, bc_mix_V1 | Qc, bc_mix_V2 |
|---|---|---|---|
| Ratio | (mgC) | (mgC) | (mgC) |
| 0.05 | 4.4 | 7.7 | 5.6 |
| 0.11 | 9.0 | 7.2 | 7.4 |
| 0.21 | 17.3 | 15.5 | 15.9 |
| 0.51 | 43.2 | 34.7 | 35.6 |
| 0.56 | 47.3 | 44.6 | 43.4 |
| 1.02 | 84.9 | 79.4 | 79.1 |
| 1.11 | 93.0 | 94.7 | 97.0 |

1-8. (canceled)

9. A process for quantifying pyrogenic carbon content present in a sample comprising an organo-mineral and a mineral matrix, charcoal and biochar comprising:
A) heating the sample under an inert atmosphere according to a first temperature sequence, with an initial temperature of between 10° and 300° C., and a final temperature of between 50° and 800° C.;
B) heating a residue of the sample resulting from the heating under an inert atmosphere under an oxidizing atmosphere according to a second temperature sequence with an initial temperature of between 10° and 300° C., and a final temperature of between 70° and 1000° C., the second temperature sequence comprising at least one thermal gradient between 1° C./min and 50° C./min, and measuring at least one amount of CO₂ released during the second temperature sequence;
C) starting, with at least one of a curve representing an evolution as a function of temperature of an amount of CO₂ released during the heating under an oxidizing atmosphere, applying a Gaussian deconvolution to the curve for determining a first and a second Gaussian centered, respectively, on a first and a second temperature, the first temperature being between 380° C. and 540° C., and the second temperature being between 500° C. and 600° C.; and
D) from surface areas of the first and second Gaussians, determining pyrogenic carbon content X₃ present in the sample with the pyrogenic carbon determined according to a formula:
$${X}_{3} = \frac{{X}_{2} - A \cdot {X}_{1}}{1 + A \cdot B}$$
in which X₁ and X₂ are carbon contents determined respectively from surface areas of the first and second Gaussians, A is a coefficient representing a proportion of the mineral matrix in the second Gaussian relative to the first Gaussian, and B is a coefficient representing a proportion of at least one of the charcoal and the biochar in the first Gaussian relative to the second Gaussian.

10. The process as claimed in claim 9, wherein the first temperature sequence comprises an isothermal steady stage of predetermined duration at an initial temperature of the first temperature sequence, followed by a thermal gradient to reach a final temperature of the first temperature sequence, the predetermined duration of the isothermal steady stage of the first temperature sequence is between 1 and 5 minutes, and the thermal gradient of the first temperature sequence is between 1° C./min and 50° C./min.

11. The process as claimed in claim 9, wherein the second temperature sequence comprises an isothermal steady stage of a predetermined duration at a temperature of between 50° and 600° C. with the predetermined duration of the isothermal steady stage of the second temperature sequence between 1 and 5 minutes.

12. The process as claimed in claim 9, wherein a sample of pure organo-mineral or pure mineral matrix is also available, in which the coefficient A of step D) is determined beforehand in which steps A) to C) are applied to a sample of pure organo-mineral or pure mineral matrix, and the coefficient A is determined by calculating a ratio between a surface area of a second Gaussian and the surface area of a first Gaussian from the sample of the pure organo-mineral or the pure mineral matrix.

13. The process as claimed in claim 9, wherein a sample of one of pure biochar and pure charcoal is available, and in which the coefficient B of step D) is determined beforehand with steps A) to C) being applied to the sample of at least one of the pure biochar and the pure charcoal, and coefficient B is determined by calculating a ratio between a surface area of the first Gaussian and the surface area of the second Gaussian from the sample of at least one of the pure biochar and the pure charcoal.

14. The process as claimed in claim 9, wherein step D) is applied with a coefficient A ranging from 0.17 to 0.73, and the coefficient B ranges from 0.10 to 4.98.

15. The process as claimed in claim 9, wherein a total mass of pyrogenic carbon Qc,bc_mix present in the sample is determined according to a formula: Qc,bc_mix=X3/C*K, wherein C is a ratio between a carbon content determined from surface area of a second Gaussian determined for a sample of at least one of the biochar and the charcoal, and a total mass of carbon in the sample of at least one of the biochar and the charcoal, and where K is a multiplicative coefficient between 12.0 and 12.5.

16. The process as claimed in claim 9, wherein a total mass of pyrogenic carbon Qc,bc_mix present in the sample is determined according to a formula: Qc,bc_mix=X3/C*K, wherein C is a ratio between 0.51 and 1.76, wherein K is a multiplication coefficient between 12.0 and 12.5.

17. The process as claimed in claim 9, wherein in step B) the gradient of the second temperature sequence is between 15° C./min and 35° C./min, in step C), the first temperature is between 415° C. and 425° C. and the second temperature is between 570° C. and 580° C.

18. The process as claimed in claim 10, wherein the first temperature is between 15° C./min and 35° C./min.

19. The process as claimed in claim 11, wherein the second temperature sequence is between 570° C. and 580° C.

20. The process as claimed in claim 14, wherein the coefficient is 0.19.
